(19) United States
(12) Patent Application Publication
(10) Pub. No.: US 2009/0281136 A1
(43) Pub. Date: Nov. 12, 2009

(54) PRASUGREL PHARMACEUTICAL FORMULATIONS

(76) Inventors: Sandeep Mhetre, Sangli (IN); Avinash Shivaji Patil, Sangli (IN); Narayanan Badri Vishwanathan, Chennai (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/437,577

(22) Filed: May 8, 2009

Related U.S. Application Data
(60) Provisional application No. 61/081,839, filed on Jul. 18, 2008.

(30) Foreign Application Priority Data
May 8, 2008 (IN) 1140/CHE/2008

(51) Int. Cl. A61K 31/4365; A61P 9/00

(52) U.S. Cl. 514/301

(57) ABSTRACT

Pharmaceutical formulations comprising prasugrel, including its pharmaceutically acceptable salts, hydrates, solvates, polymorphs, and mixtures thereof. Also described are processes for preparing the stable formulations and their methods of use.

Aspects of the present invention relate to pharmaceutical formulations comprising prasugrel, or pharmaceutically acceptable salts thereof. More specifically, aspects of the present invention relate to stable pharmaceutical formulations comprising prasugrel, or pharmaceutically acceptable salts thereof, for therapeutic purposes, and methods of preparing the same.

Platelet activation and subsequent platelet aggregation play an essential role in the pathogenesis of cardiovascular, cerebrovascular and peripheral vascular diseases. Upon vascular injury, adenosine diphosphate (ADP), a potent platelet activator, is released into the blood stream from damaged cells and activated platelets, which in turn acts on other platelets. ADP induces a number of responses in platelets, including shape change from discs to spheres, and aggregation and secretion of granule contents. Thienopyridine derivatives such as ticlopidine and clopidogrel are orally active inhibitors of ADP-induced platelet aggregation with a slow onset and long duration of action. Several lines of preclinical and clinical investigations have clearly demonstrated the efficacy of ticlopidine and clopidogrel, and these agents have been used for the treatment of thrombosis and related diseases. Clopidogrel in particular has found widespread use, as compared to the older ticlopidine.

Prasugrel is a thienopyridine derivative, and an ADP receptor antagonist. It is a potent inhibitor of ADP-mediated platelet aggregation in vivo. It produces more potent platelet inhibition and a rapid onset of action and may provide a superior therapeutic alternative to clopidogrel.

Prasugrel has the chemical names 2-acetoxy-5-α-cyclopropylcarbonyl-2-fluorobenzyl)-4,5,6,7-tetrahydrothieno[3,2-c]pyridine, or 5-[2-cyclopropyl-1-(2-fluorophenyl)-2-oxoethyl]-4,5,6,7-tetrahydrothieno[3,2-c]pyridin-2-yl acetate, a molecular formula C₂₀H₂₀FNO₃S, and is represented by structural Formula I.

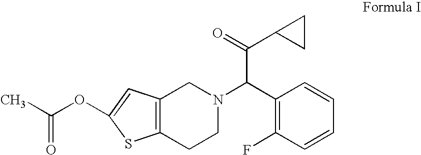

A product containing prasugrel recently was approved for marketing in Europe. Daiichi Sankyo Co. and its partner Eli Lilly and Company have submitted a marketing application in the U.S. for prasugrel in the treatment of patients with acute coronary syndrome who are managed with percutaneous coronary intervention.

U.S. Pat. No. 5,288,726 discloses and claims tetrahydrothienopyridine derivatives including prasugrel. This patent document discloses the use of these compounds for treatment and prophylaxis of thrombosis and embolisms.

U.S. Patent Application Publication No. 2004/0024013 A1 describes a method of treating vascular diseases by administering prasugrel or a pharmaceutically acceptable salt thereof, and aspirin.

International Application Publication No. WO 2006/138317 describes a dosage regimen for treating vascular disease in a human comprising administering a loading dose of about 30 mg to 70 mg of prasugrel or a pharmaceutically acceptable salt thereof, and thereafter administering a maintenance daily dosage regimen of about 7.5 mg to 15 mg of prasugrel or a pharmaceutically acceptable salt thereof.

U.S. Patent Application Publication No. 2006/0217351 A1 describes a method of treating cardiovascular diseases comprising administering prasugrel, in conjunction with coronary and non-coronary intervention procedures.

U.S. Pat. No. 6,693,115 discloses and claims the hydrochloric acid and maleic acid salts of prasugrel. The HCl and maleate salt forms provide improvements in their efficacy and stability profiles compared to other salts and also compared to the free base molecule. However, prolonged exposure of prasugrel to air and moisture results in degradation. Hence, there is a need for improvements in the stability, shelf life and therefore long term efficacy of individual doses of prasugrel.

International Application Publication No. WO 2006/135605 describes a formulation comprising a therapeutically effective amount of prasugrel hydrochloride packaged in an air and moisture impervious blister package, with an inert gas atmosphere, to improve the stability and shelf life of prasugrel.

There remains a need for stable pharmaceutical formulations comprising prasugrel or a pharmaceutically acceptable salt thereof.

### SUMMARY

The present invention relates to stable pharmaceutical formulations comprising prasugrel or pharmaceutically acceptable salts thereof for therapeutic purposes, and methods of preparing the same.

In an embodiment, the invention includes pharmaceutical formulations comprising prasugrel or a pharmaceutically acceptable salt thereof and at least one antioxidant.

In an embodiment, the invention includes pharmaceutical formulations comprising prasugrel or a pharmaceutically acceptable salt thereof and at least one antioxidant, wherein said antioxidant is present in concentrations about 0.05 to about 10% by weight of said formulation.

In an embodiment, the invention includes pharmaceutical formulations comprising prasugrel or a pharmaceutically acceptable salt thereof and one or more pharmaceutically acceptable excipients, wherein one or more excipients are of low moisture grade.

In an embodiment, the invention includes pharmaceutical formulations comprising: (a) prasugrel or a pharmaceutically acceptable salt thereof; (b) at least one antioxidant; and (c) one or more pharmaceutically acceptable excipients, wherein one or more excipients are of low moisture grade.

In an embodiment, the invention includes pharmaceutical formulations comprising prasugrel or a pharmaceutically acceptable salt thereof and one or more pharmaceutically acceptable excipients, wherein said formulation is packaged together with one or more of a desiccant and an oxygen scavenger.

In another embodiment, the invention includes methods of preparing the pharmaceutical formulations of the present invention.

In a further embodiment, the invention includes methods of treating patients suffering from thrombotic disorders using pharmaceutical formulations of the present invention.

### DETAILED DESCRIPTION

In aspects, the present invention relates to pharmaceutical formulations comprising prasugrel or pharmaceutically acceptable salts thereof for oral administration, and methods for preparing the same.

As used herein the term “prasugrel” means a compound including, but not limited to, any of the drug compound prasugrel, its pharmaceutically acceptable salts, their prodrugs, the active metabolites and the prodrugs thereof, their enantiomers, racemic mixtures, mixtures of isomers, and their polymorphs, solvates and hydrates thereof.

The terms “pharmaceutically acceptable salt” refers to salts including, but not limited to: hydrohalogen acid salts such as the hydrofluoride, hydrochloride, hydrobromide and hydroiodide; the nitrate; the perchlorate; the sulfate; the phosphate; a C₁-C₄ alkanesulfonate, optionally substituted by halogens, such as methanesulfonate, trifluoromethanesulfonate, and ethanesulfonate; a C₆-C₁₀ arylsulfonate, optionally substituted by C₁-C₄ alkyl groups, such as benzenesulfonate and p-toluenesulfonate; a C₁-C₆ aliphatic acid salt such as acetate, malate, fumarate, succinate, citrate, tartarate, oxalate and maleate; and an amino acid salt such as the glycine salt, lysine salt, arginine salt, ornithine salt, glutamic acid salt and aspartic acid salt.

According to the present invention, prasugrel and its salts can be used in any crystalline form, or in amorphous form, or in combinations thereof.

The terms “excipient” and “pharmaceutically acceptable excipient” mean a component of a pharmaceutical product that is not a pharmacologically active ingredient, such as a filler, diluent, binder, carrier, etc. Pharmaceutical formulations frequently contain two or more excipients. The excipients that are useful in preparing a pharmaceutical formulation are generally safe, non-toxic and neither biologically nor otherwise undesirable, and are acceptable for veterinary use as well as human pharmaceutical use.

The term “stabilizing agent” means any substance that is used to prevent prasugrel physical or chemical degradation, such as an antioxidant, desiccant, and a preservative.

The term “antioxidant” means any substance that is capable of slowing or preventing an oxidation reaction (due to contact with oxygen) such as that caused by free radicals. Free radicals are highly reactive chemicals that attack molecules by capturing electrons and thus modifying chemical structures. Antioxidants terminate these chain reactions by removing free radical intermediates, and inhibit other oxidation reactions by being oxidized themselves.

The term “desiccant” means any hygroscopic substance that induces or sustains a state of dryness (desiccation) in its local vicinity in a closed container. The desiccant can be supplied in the form of a sachet, packet, cartridge or canister. Commonly encountered pre-packaged desiccants are solids, and work through absorption or adsorption of water, or a combination of the two. A pre-packaged desiccant (such as in a pouch) is commonly used to remove excessive humidity that would normally degrade or even destroy products that are sensitive to moisture.

The term “oxygen scavenger” or “oxygen absorber” means any chemical substance that works to maintain an atmosphere that is reduced in its oxygen content. There are two major sources of oxygen in permeable bottles typically used in the pharmaceutical industry: (1) oxygen in the headspace; and (2) oxygen that permeates through the container walls. The amount of oxygen contributed by the two sources will vary with the size and shape of the bottle, and the means by which the top is sealed. The headspace oxygen will also depend on the fill volume in the bottle. To be effective, an oxygen absorber is incorporated into the bottle such that the atmosphere surrounding an oxygen-sensitive drug has sufficient contact with the oxygen-absorber to remove at least a portion of the oxygen from the air, to stop or retard the degradation process.

A pharmaceutical formulation according to the present invention can be presented in forms such as tablets, capsules, granules, spheroids, beads, pellets, mini-tablets, multilayered tablets, powders, sachets, gels, dispersions, solutions or suspensions.

The stability of prasugrel and the pharmaceutical formulations of prasugrel will be affected by factors including age (length of storage after manufacture), and storage conditions, such as for example, temperature and relative humidity. The proper formulation and storage conditions ensure an extended shelf life during which the potency of the formulations will remain within specification limits, thereby ensuring the chemical and pharmacodynamic integrity of the formulations administered to patients.

Studies have shown that the hydrochloride and maleate salt forms provide improved efficacy and stability profiles compared to other salts and also compared to the free base molecule (see U.S. Pat. No. 6,693,115). Another study revealed that stored tablets containing prasugrel degrade by both hydrolytic and oxidative pathways. There are crossovers between these degradation pathways wherein intermediates or products of certain steps in one pathway may interconvert or be kinetically accelerated or hindered by the concentration of product (or intermediate), air or moisture from the environment or the other pathway (see International Application Publication No. WO 2006/135605). This study involved a formulation comprising a therapeutically effective amount of prasugrel hydrochloride packaged in an air and moisture impervious gas-inerted blister package to improve the stability and shelf life of prasugrel. Despite the improvements associated with these studies, there remains a need for alternate approaches to prepare stable pharmaceutical formulations comprising prasugrel.

In an embodiment, the invention includes pharmaceutical formulations comprising prasugrel or a pharmaceutically acceptable salt thereof and at least one antioxidant.

In an embodiment, the invention includes pharmaceutical formulations comprising prasugrel or a pharmaceutically acceptable salt thereof and at least one antioxidant, wherein said antioxidant is present in a concentration of about 0.05 to about 10% by weight of said formulation.

Useful antioxidants include butylated hydroxyanisole (BHA), butylated hydroxytoluene (BHT), ascorbic acid or a salt thereof (e.g., a sodium salt, a calcium salt, a magnesium salt, a potassium salt, a basic amino acid salt, or a meglumine salt), sodium nitrite, sodium hydrogen sulfite, sodium sulfite, a salt of edetic acid (e.g., a sodium salt, a potassium salt, or a calcium salt), erithorbic acid, cysteine hydrochloride, citric acid, cysteine, potassium dichloroisocyanurate, sodium thioglycolate, thioglycerol, sodium formaldehyde sulfoxylate, sodium pyrosulfite, 1,3-butylene glycol, propyl gallate, carotenoids, retinol and esters thereof, a tocopherol or its derivatives, and mixtures thereof. Other antioxidants or chelating agents and such other additives as desired to enhance the stability of prasugrel are included within the scope of this invention without limitation.

The formulations of the present invention can be prepared by combining prasugrel or a salt thereof with one or more antioxidants, and optionally with other pharmaceutically acceptable excipients, and compounding to form a pharmaceutical formulation, e.g., compressing into tablets or filling into suitable capsule shells, using techniques known to those having skill in the art. These formulations can be packaged in bottles along with one or more desiccants for further protection from degradation.

In an embodiment, the invention includes pharmaceutical formulations comprising prasugrel or a pharmaceutically acceptable salt thereof and at least one antioxidant, wherein said formulation is packaged in bottles along with one or more desiccants and/or oxygen scavengers.

In an aspect, the invention includes stabilized premixes comprising prasugrel or a pharmaceutically acceptable salt thereof and at least one antioxidant, processes for their preparation and pharmaceutical formulations comprising them.

In an aspect, the invention includes processes for preparing stabilized pre-mixes comprising prasugrel or a pharmaceutically acceptable salt thereof and at least one antioxidant, wherein an embodiment comprises: (a) providing a solution of prasugrel and at least one antioxidant; and (b) removing the solvent.

Solvents that may be used for dissolving prasugrel include, but are not limited to: alcohols such as methanol, ethanol, isopropyl alcohol, 1-propanol, 2-propanol, 1-butanol, 2-butanol, and t-butyl alcohol; halogenated hydrocarbons such as dichloromethane, 1,2-dichloroethane, chloroform and carbon tetrachloride; ketones such as acetone, ethyl methyl ketone, and methyl isobutyl ketone; esters such as ethyl acetate, n-propyl acetate, n-butyl acetate and t-butyl acetate; ethers such as diethyl ether, dimethyl ether, diisopropyl ether, methyl t-butyl ether and 1,4-dioxane; nitriles such as acetonitrile and propionitrile; water; and mixtures thereof.

During preparation of pre-mixes the solvent may be removed by techniques known in art, including, but not limited to, distillation, evaporation, oven drying, tray drying, rotational drying (such as using a Buchi Rotovapor), spray drying, freeze drying, fluidized bed drying, flash drying, spin flash drying, thin film drying, and the like.

A solid pre-mix of prasugrel or a pharmaceutically acceptable salt thereof and at least one antioxidant provides a product with desired characteristics like stability and exhibits good processing characteristics, and can be easily and conveniently processed into pharmaceutical formulations (such as, for example, tablets, capsules, and the like).

In another aspect, stable pharmaceutical formulations comprising prasugrel can be prepared by combining the drug or its pre-mix with one or more pharmaceutically acceptable excipients such as diluents, binders, disintegrants, lubricants etc., in such a way that they have a low moisture content to protect prasugrel from degradation. The low moisture grade excipients are combined with prasugrel or a salt thereof, and compounded to form a pharmaceutical formulation, i.e., they are compressed into tablets or placed into suitable capsule shells, using techniques known to those having skill in the art. These formulations optionally contain antioxidants to protect prasugrel from oxidation. The final formulations can be packaged in closed containers such as bottles, along with one or more of a desiccant and an oxygen scavenger.

In an embodiment, the invention includes pharmaceutical formulations comprising prasugrel or a pharmaceutically acceptable salt thereof and one or more pharmaceutically acceptable excipients, wherein said one or more excipients are of low moisture grade.

In another embodiment, the invention includes pharmaceutical formulations comprising: (a) prasugrel or a pharmaceutically acceptable salt thereof; (b) at least one antioxidant; and (c) one or more pharmaceutically acceptable excipients; wherein said one or more excipients are of low moisture grade.

In another embodiment, the invention includes pharmaceutical formulations comprising prasugrel or a pharmaceutically acceptable salt thereof and one or more low moisture grade pharmaceutically acceptable excipients, wherein said formulation is packaged in closed containers along with one or more of a desiccant and an oxygen scavengers.

Generally, the moisture content of solid formulations will not exceed about 5 percent by weight. This can desirably be achieved, in part, by the use of one or more excipients having low moisture contents.

In further embodiments, the invention includes pharmaceutical formulations comprising prasugrel or a pharmaceutically acceptable salt thereof and one or more pharmaceutically acceptable excipients, wherein said formulation is packaged in closed containers along with one or more of a desiccant and an oxygen scavenger.

The desiccants for use in the practice of the invention can be any available desiccants, which include those commonly used in the pharmaceutical industry, which have adequate capacity to handle the combination of moisture ingress through the bottle and any moisture given off by a self-activating oxygen absorber. Suitable desiccants are discussed by R. L. Dobson, Journal of Packaging Technology, Vol. 1, pp. 127-131 (1987). Various useful desiccants include, but are not limited to, silica gel, calcium sulfate, calcium chloride, montmorillonite clay, and molecular sieves. The desiccant can be supplied in the form of a sachet, packet, cartridge or canister. Examples of commercially available desiccants include, but are not limited to, SorBit™ (a canister of silica gel supplied by Sud-Chemie Performance Packaging, Belen, N. Mex., USA), Desi Pak® (bentonite clay), Sorb-It® (silica gel), Getter Pak® (activated carbon), 2-in-1 Pak® (silica gel or bentonite clay and activated carbon) and Tri-Sorb® (molecular sieve), all of which are supplied by Texas Technologies Inc., Texas USA).

A molecular sieve is a material containing tiny pores of a precise and uniform size that is used as an adsorbent for gases and liquids. Molecular sieves often consist of aluminosilicate minerals, clays, porous glasses, microporous charcoals, zeolites, active carbons, or synthetic compounds that have open structures into which small molecules, such as nitrogen and water can diffuse. Molecules small enough to enter the pores are adsorbed while larger molecules are not. It is different from a common filter in that it operates on a molecular level. For instance, a water molecule may be small enough to pass through while larger molecules are not. Because of this, they often function as desiccants. A dried molecular sieve can adsorb water up to 22% of its own weight.

Suitable oxygen absorbers for use in the practice of the invention include those commonly used in the pharmaceutical industry. These include but are not limited to metal-based absorbers such as particulate iron (e.g., hydrogen-reduced iron, electrolytically reduced iron, atomized iron, and milled pulverized iron powders), copper powder, and zinc powder. The oxygen absorber may be provided in a sachet, packet, cartridge, canister or any other means of containing the absorber such that the absorber is physically separated from products placed in a container and has sufficient oxygen permeability to remove at least a portion of the oxygen in the air within the container. The amount of oxygen-absorber added will depend upon the volume of air surrounding the product, the permeability of the container, the oxidation potential of the drug, and the means by which the oxygen-absorber is incorporated into the construction. Useful commercially available oxygen-absorbers include FreshPax® Packets and Strips and StabilOx® oxygen and moisture management packets (both available from Multisorb Technologies Inc., Buffalo, N.Y., USA), Ageless™ and ZPTJ™ sachets (both available from Mitsubishi Gas Corporation, Tokyo, Japan), O-Busterm (available from Hsiao Sung Non-Oxygen Chemical Co., Ltd., Taiwan, R.O.C.), Bioka™ Oxygen Absorber (available from Bioka Ltd., Kantvik, Finland), and the like.

The different physicochemical properties of the active ingredient and as well as of excipients are to be considered, as these properties affect the process and formulation properties of the compound. Various important physicochemical properties include but are not limited to particle sizes, density (bulk density and tapped density), compressibility index, Hausner's ratio, angle of repose, etc. Particle sizes of active pharmaceutical ingredient can affect the solid dosage form in numerous ways. For example, content uniformity (CU) of pharmaceutical dosage units can be affected by particle size and size distribution. This will be even more critical for low-dose drugs and satisfactory dosage units of low doses cannot be manufactured from a drug that does not meet certain particle size and size distribution specifications. Also particle sizes play an important role in dissolution of active ingredient form the final dosage form for certain drugs like prasugrel because of their poor solubility. Hence, these physicochemical properties not only affect the processes of the preparing the pharmaceutical formulations but also affect the performance of the pharmaceutical product both in vitro and in vivo.

The D₁₀, D₅₀, and D₉₀ values are useful ways for indicating a particle size distribution. D₉₀ is a size value where at least 90 percent of the particles have a size smaller than the stated value. Likewise D₁₀ refers to 10 percent of the particles having a size smaller than the stated value. D₅₀ refers to at least 50 percent of the particles having a size smaller than the stated value and D_{[4,3]} value refers to a mean particle size. Methods for determining D₁₀, D₅₀ D₉₀ and D_{[4,3]} include those using laser light diffraction with equipment sold by Malvern Instruments Ltd., Malvern, Worcestershire, United Kingdom.

In certain embodiments, the pharmaceutical formulations of the present invention optionally include excipients, which include without limitation one or any combination of diluents, binders, disintegrants, lubricants, glidants, and other additives that are commonly used in solid pharmaceutical dosage form preparations.

### Diluents:

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different starches include but are not limited to maize starch, potato starch, rice starch, wheat starch, corn starch (UNI-PURE DW-L™ low moisture grade from National Starch and Chemical Company, New Jersey USA), pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH101, PH102, PH112, PH113, PH200, PH301, PH302 and PH-F20. Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include but are not limited to hydroxypropylcelluloses, also called HPC (Klucel™ LF, Klucel EXF) and useful in various grades, hydroxypropyl methylcelluloses, also called hypromelloses or HPMC (Methocel™) and useful in various grades, polyvinylpyrrolidones or povidones (such as grades PVP-K25, PVP-K29, PVP-K30, and PVP-K90), Plasdone™ S 630 (copovidone), powdered acacia, gelatin, guar gum, carbomer (Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Lubricants:

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow of powder blends and minimize dosage form weight variation, can be used. Useful glidants include but are not limited to silicone dioxide, talc and combinations thereof.

### Coloring Agents:

Coloring agents can be used to color code the formulations, for example, to indicate the type and dosage of the therapeutic agent therein. Suitable coloring agents include, without limitation, natural and/or artificial compounds such as FD&C coloring agents, natural juice concentrates, pigments such as titanium oxide, silicon dioxide, iron oxides, zinc oxide, combinations thereof, and the like.

### Solvents:

Various solvents can be used in the processes of preparation of pharmaceutical formulations of the present invention including, but not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

In embodiments, the pharmaceutical formulations of the present invention optionally have one or more coatings including seal coatings, elegant coatings and delayed release coatings. Additional excipients such as film forming polymers, plasticizers, antiadherents, opacifiers and optionally colorants, pigments, antifoam agents, polishing agents can be used in coating operations.

### Film-Forming Agents:

Various film-forming agents include but are not limited to cellulose derivatives such as soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, etc., insoluble cellulose derivative such as ethylcelluloses and the like, dextrins, starches and starch derivatives, polymers based on carbohydrates and derivatives thereof, natural gums such as gum Arabic, xanthans, alginates, polyacrylic acid, polyvinyl alcohols, polyvinyl acetates, polyvinylpyrrolidones, polymethacrylates and derivatives thereof (Eudragit™ products), chitosan and derivatives thereof, shellac and derivatives thereof, waxes, and fat substances. Useful enteric coating materials include but are not limited to materials such as cellulosic polymers like cellulose acetate phthalates, cellulose acetate trimellitates, hydroxypropyl methylcellulose phthalates, polyvinyl acetate phthalates, etc., methacrylic acid polymers and copolymers (Eudragit™), and the like, and mixtures thereof.

Some excipients are used as adjuvants for coating processes, including excipients such as plasticizers, opacifiers, antiadhesives, polishing agents, etc.

Various useful plasticizers include, but are not limited to, castor oil, diacetylated monoglycerides, dibutyl sebacate, diethyl phthalate, glycerin, polyethylene glycol, propylene glycol, triacetin, triethyl citrate, and mixtures thereof. An opacifier like titanium dioxide may also be present in an amount ranging from about 10% (w/w) to about 20% (w/w), based on the total weight of the coating.

Antiadhesives are frequently used in the film coating process to avoid sticking effects during film formation and drying. An example of an antiadhesive for this purpose is talc.

Suitable polishing agents include polyethylene glycols of various molecular weights or mixtures thereof, talc, surfactants (e.g., glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

In addition to the above coating ingredients, sometimes pre-formulated coating products such as those sold as OPADRY™ AMB (Aqueous Moisture Barrier) supplied by Colorcon or TABCOAT™ can be used. OPADRY formulations generally comprise polymer, plasticizer and, if desired, pigment in a dry concentrate. OPADRY products produce attractive, elegant coatings on a variety of tablet cores and can be used in both aqueous and organic coating procedures. Preformulated coating products generally require only dispersion in a liquid before use.

In another aspect, stable pharmaceutical formulations comprising prasugrel can be prepared by coating tablets comprising prasugrel, with gelatin. Gelatin coatings with adequate thickness provide protection for the formulations against degradation during storage. In addition to imparting stability, gelatin coatings facilitate swallowing and gelatin coated tablets are more tamper-evident. These coatings can be applied to the tablets through a dip coating technique, and other techniques that are known to those having skill in the art.

In an embodiment, the invention includes pharmaceutical formulations comprising prasugrel or a pharmaceutically acceptable salt thereof, wherein the formulations are gelatin coated.

Alternatively, soft gelatin capsules comprising prasugrel can be prepared to protect prasugrel from degradation. In this approach the active ingredient, prasugrel or its pharmaceutically acceptable salt, is dissolved or dispersed in a suitable liquid vehicle along with optional excipients and filled into soft gelatin capsules using methods known in the art.

In an embodiment, the invention includes soft gelatin capsules having a capsule shell comprising gelatin and at least one plasticizer, and a capsule filling containing a liquid vehicle, having dissolved or dispersed therein prasugrel or its pharmaceutically acceptable salt along with optional excipients.

Suitable liquid vehicles for use in the practice of the invention include but are not limited to oils, propylene glycol, polysorbates and polyethylene glycols. The vehicles used for the oily matrix should be inert, non-toxic, biocompatible, easy to handle, easily available and inexpensive. The vehicle chosen should also provide appropriate consistency and stability in the final product. Suitable oils include corn oil, sesame oil, peanut oil, bean oil, almond oil, peach kernel oil, cotton seed oil, sunflower seed oil, castor oil, olive oil, linseed oil, tung oil, angelica essential oil, soybean oil, safflower oil, canola oil, macadamia nut oil, arachis oil, wheat germ oil, rice bran oil, and mixtures thereof.

The formulations of the present invention may be prepared using process steps including one or more of wet granulation, melt granulation, dry granulation such as slugging or compaction, direct compression, and various coating processes, and can be formulated into dosage forms including tablets and capsules.

Equipment suitable for processing the pharmaceutical formulations of the present invention include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans; tray dryers, fluid bed dryers, rotary cone vacuum dryers, and the like, multimills, fluid energy mills, ball mills, colloid mills, roller mills, hammer mills, and the like, equipped with a suitable screen.

In other embodiments, the invention includes methods of preparing the pharmaceutical formulations of the present invention.

In an aspect the present invention provides processes for preparing formulations comprising prasugrel or a salt thereof, wherein an embodiment of a process comprises:

i) sifting prasugrel and an antioxidant through a sieve and mixing the sifted materials;

ii) sifting diluents, binders, glidants, lubricants and any other desired excipients through a sieve;

iii) adding the sifted excipients, except lubricants, to the blend of step (i) and blending;

iv) adding lubricants and blending;

v) compressing the final blend into tablets or filling into empty hard gelatin capsules;

vi) optionally, coating tablets; and

vii) packaging the capsules or tablets in closed bottles together with a desiccant.

In an aspect the present invention provides processes for preparing a formulation comprising prasugrel or a salt thereof, wherein an embodiment of a process comprises:

i) sifting prasugrel, diluents, binders, glidants, lubricants and any other desired excipients through a sieve;

ii) blending the sifted excipients, except lubricants by geometric mixing:

iii) adding lubricants and blending;

iv) compressing the final blend into tablets or filling into empty hard gelatin capsule;

v) optionally, coating tablets; and

vi) packaging the capsules or tablets in closed bottles together with a desiccant and a oxygen scavenger.

Dosage forms prepared as above can be subjected to in vitro dissolution evaluations such as that according to Test 711 “Dissolution” in United States Pharmacopoeia 29, United States Pharmacopeial Convention, Inc., Rockville, Md., 2005 (“USP”) to determine the rate at which the active substance is released from the dosage forms. The amounts of active substance and impurities in solutions are conveniently measured using techniques such as high performance liquid chromatography (HPLC).

In some embodiments, the invention includes use of packaging materials such as containers and closures of high-density polyethylene (HDPE), low-density polyethylene (LDPE) and or polypropylene and/or glass, glassine foil, aluminium pouches, and blisters or strips composed of aluminium or high-density polypropylene, polyvinyl chloride, polyvinylidene dichloride, etc.

In further embodiments the invention includes methods of treating patients suffering from thrombotic disorders using pharmaceutical formulations of the present invention. Thrombotic disorders can be due to the formation or presence of a blood clot within a blood vessel due to prior and acute myocardial infarction, unstable and stable angina, acute reocclusion after percutaneous transluminal coronary angioplasty (PTCA), restenosis, thrombotic stroke, prior transient ischemic attack (TIA) and reversible ischemic neurological deficit (RIND).

Pharmaceutical formulations of the present invention can optionally be administered together with one, or more than one, other therapeutic agents in the treatment of thrombotic disorders including, but not limited to, salicylates such as aspirin, angiotensin 11 receptor antagonists such as candesartan, valsartan, eprosartan, losartan, irbesartan, saprisartan, zolasartan, saralasin, telmisartan, and tasosartan, isoteoline, HMG CoA reductase inhibitors such as atorvastatin, cerivastatin, fluvastatin, lovastatin, pravastatin, rosuvastatin, simvastatin, pitvastatin, fluindostatin, mevastatin, velostatin and dalvastatin, and any pharmaceutically acceptable salts, solvates, hydrates, enantiomers thereof. The useful therapeutic agents are well known to those skilled in the art, and the use of any of them falls within the scope of the invention.

The pharmaceutical dosage forms of the present invention are intended for oral administration to a patient in need thereof.

Certain specific aspects and embodiments of the invention will be explained in more detail with reference to the following examples, being provided only for purposes of illustration, and it is to be understood that the present invention should not be deemed to be limited thereto.

### Example 1

### Drug-Excipient Compatibility Study

Prasugrel hydrochloride and mixtures with excipients in the weight ratios stated below are mixed well and sifted through an ASTM #40 mesh sieve. The mixture is placed into open glass vials, stored at 40° C. and 75% relative humidity for four weeks, and samples are analyzed at intervals to evaluate the extent of impurity formation. Values for impurities in the table below are calculated as percent by weight of the original prasugrel content.

| | | Total Impurities | | | | |
|---|---|---|---|---|---|---|
| | | | 1 | 2 | 3 | 4 |
| Mixture | Drug:Excipient | Initial | Week | Weeks | Weeks | Weeks |
| Prasugrel | — | 0.6301 | 0.6547 | 0.6982 | 0.7488 | 0.8998 |
| Prasugrel + Sodium | 1:0.5 | 0.73 | 0.85 | 0.74 | 0.98 | 1.11 |
| bisulfite | | | | | | |
| Prasugrel + Stabilox* | — | 0.6301 | 0.6352 | 0.913 | 0.7622 | 0.911 |
| Prasugrel + Stearic | 1:4 | 0.7467 | 0.7869 | 0.32 | — | — |
| acid | | | | | | |

*Stabilox ™ reduces oxygen levels and maintains a specific equilibrium relative humidity (ERH) level; one packet is placed into each vial.

The above data show that prasugrel has acceptable stability in the presence of the excipients studied under accelerated stability testing conditions.

### Example 2

### Prasugrel 60 mg Tablets

| Ingredient | mg/Tablet |
|---|---|
| Prasugrel hydrochloride | 65.88* |
| Fumed silica | 6 |
| Sodium bisulfite | 1.5 |
| Lactose monohydrate | 190.62 |
| Croscarmellose sodium | 12 |
| Hydroxypropyl methylcellulose 3 cps | 15 |
| Fumed silica | 6 |
| Magnesium stearate | 3 |

Coating

| | |
|---|---|
| OPADRY AMB** | 9 |
| Water*** | 81 |

*Equivalent to 60 mg prasugrel. Particle size distribution of Prasugrel hydrochloride used in this example: D₁₀ < 1.11 μm; D₉₀ < 2.36 μm; D₉₀ < 4.8 μm
**OPADRY AMB contains partially hydrolyzed polyvinyl alcohol, talc, lecithin, and xanthan gum, supplied by Colorcon.
***Evaporates during processing.

Manufacturing Process:

1. Prasugrel hydrochloride, sodium bisulfite and fumed silica (first quantity) are sifted through a #60 mesh sieve and mixed thoroughly.

2. The blend of step 1 is added to lactose monohydrate, croscarmellose sodium and hydroxypropyl methylcellulose geometrically, and mixed thoroughly.

3. Fumed silica (second quantity) and magnesium stearate are sifted through a #60 mesh sieve, added to the blend of step 2, and mixed thoroughly.

4. The blend of step 3 is compressed into tablets.

5. Tablets of step 4 are coated with Opadry AMB dispersion in water.

6. Coated tablets are packaged in a closed HDPE bottle containing a Stabilox packet.

The tablets are analyzed for drug content, impurities and drug dissolution characteristics, before and after storage at 40° C. and 75% relative humidity conditions for 1 week. Dissolution testing is also conducted, using USP apparatus 2, 900 ml of 0.1 N HCl, and rotation at 50 rpm. The data are given below.

| Parameter | Initial | 1 Week |
|---|---|---|
| Prasugrel Assay (% of label content) | 85 | — |
| Total Impurities (% of label prasugrel | 3.0507 | 3.1713 |
| content) | | |

Dissolution (cumulative % of drug released)

| | | |
|---|---|---|
| 15 minutes | 51 | — |
| 30 minutes | 79 | — |

### Examples 3-5

### Prasugrel Tablets

| | mg/Tablet | | |
|---|---|---|---|
| Ingredient | Example 3 | Example 4 | Example 5 |
| Prasugrel hydrochloride | 16.47* | 32.94** | 65.88*** |
| Anhydrous lactose | 181.17 | 164.7 | 131.76 |
| Hydroxypropyl methylcellulose | 10.5 | 10.5 | 10.5 |
| 3 CPS | | | |
| Croscarmellose sodium | 12 | 12 | 12 |
| Anhydrous lactose | 64.71 | 64.71 | 64.71 |
| Hydroxypropyl methylcellulose | 4.5 | 4.5 | 4.5 |
| 3 CPS | | | |
| Sodium bisulfite | 1.65 | 1.65 | 1.65 |
| Isopropyl alcohol‡ | 25 | 25 | 25 |
| Colloidal silicon dioxide | 6 | 6 | 6 |
| Magnesium stearate | 3 | 3 | 3 |

Coating

| | | | |
|---|---|---|---|
| OPADRY AMB | 9 | 9 | 9 |
| Water‡ | 81 | 81 | 81 |

* Equivalent to 15 mg prasugrel.
**Equivalent to 30 mg prasugrel.
***Equivalent to 60 mg prasugrel.
‡Evaporates during processing.

Manufacturing Process:

1. Mix prasugrel hydrochloride, lactose (first quantity), hydroxypropyl methylcellulose (first quantity) and croscarmellose sodium thoroughly.

2. Compact the blend of step 1 using a roller compactor.

3. Mill the compacted material of step 2 and sift through a #30 mesh sieve.

4. Mix lactose (second quantity), hydroxypropyl methylcellulose (second quantity) and sodium bisulfite thoroughly and granulate with isopropyl alcohol.

5. Dry the granules of step 4 and sift through a #30 mesh sieve.

6. Mix the blend of step 3 with the granules of step 5 thoroughly.

7. Sift colloidal silicon dioxide and magnesium stearate through a #60 mesh sieve, add to the blend of step 6, and mix thoroughly.

8. Compress the blend of step 7 into tablets.

9. Coat the tablets of step 8 with Opadry AMB dispersion.

10. Package the tablets of step 9 in a closed HDPE bottle containing SorBit™ (a packet of silica gel supplied by Sud-Chemie Performance Packaging, Belen, N. Mex., USA) and a Stabilox packet.

### Examples 6-8

### Prasugrel Tablets

| | mg/Tablet | | |
|---|---|---|---|
| Ingredient | Example 6 | Example 7 | Example 8 |
| Prasugrel hydrochloride | 16.47 | 32.94 | 65.88 |
| Stearic acid | 131.76 | 131.76 | 131.76 |
| Methanol* | 6 | 6 | 6 |
| Sodium bisulfite | 0.3 | 0.3 | 0.3 |
| Anhydrous lactose | 130.47 | 114 | 81.06 |
| Croscarmellose sodium | 12 | 12 | 12 |
| Fumed silica | 6 | 6 | 6 |
| Magnesium stearate | 3 | 3 | 3 |

Coating

| | | | |
|---|---|---|---|
| OPADRY AMB | 9 | 9 | 9 |
| Water* | 81 | 81 | 81 |

*Evaporates during processing.

Manufacturing Process:

1. Dissolve prasugrel hydrochloride and stearic acid in methanol, and evaporate to dryness at 60° C.

2. Add the dried material of step 1 to sodium bisulfite, lactose and croscarmellose sodium geometrically, and mix thoroughly.

3. Sift fumed silica and magnesium stearate through a #60 mesh sieve, add to the blend of step 2, and mix thoroughly.

4. Compress the blend of step 3 into tablets.

5. Coat the tablets of step 4 with Opadry AMB dispersion.

6. Package the tablets of step 5 in a closed HDPE bottle containing a Sorb-It packet.

### Examples 9-11

### Prasugrel Tablets

| | mg/Tablet | | |
|---|---|---|---|
| Ingredient | Example 9 | Example 10 | Example 11 |
| Prasugrel hydrochloride | 16.47 | 32.94 | 65.88 |
| Stearic acid | 131.76 | 131.76 | 131.76 |
| Hydroxypropyl methylcellulose | 4.5 | 4.5 | 4.5 |
| 3 CPS | | | |
| Methanol* | 6 | 6 | 6 |
| Anhydrous lactose | 125.97 | 109.5 | 76.56 |
| Sodium bisulfite | 0.3 | 0.3 | 0.3 |
| Croscarmellose sodium | 12 | 12 | 12 |
| Fumed silica | 6 | 6 | 6 |
| Magnesium stearate | 3 | 3 | 3 |

Coating

| | | | |
|---|---|---|---|
| OPADRY AMB | 9 | 9 | 9 |
| Water* | 81 | 81 | 81 |

*Evaporates during processing.

Manufacturing Process:

1. Dissolve prasugrel hydrochloride, stearic acid, and hydroxypropyl methylcellulose in methanol.

2. Spray the solution of step 1 onto lactose and dry to form a free-flowing solid.

3. Add sodium bisulfite and croscarmellose sodium to the material of step 2 and mix thoroughly.

4. Sift fumed silica and magnesium stearate through a #60 mesh sieve, add to the blend of step 3, and mix thoroughly.

5. Compress the blend of step 4 into tablets.

6. Coat the tablets of step 5 with Opadry AMB dispersion in water.

7. Package the tablets of step 5 in a closed HDPE bottle containing a Sorb-It packet.

### Example 12

### Prasugrel Tablets

| Ingredient | mg/Tablet |
|---|---|
| Prasugrel hydrochloride | 65.88 |
| Fumed silica (Aerosil 200) | 6 |
| Sodium bisulfite§ | 1.5 |
| Lactose monohydrate | 190.62 |
| Croscarmellose sodium | 12 |
| HPMC 3 cps | 15 |
| Fumed silica | 6 |
| Magnesium stearate | 3 |

Coating

| | |
|---|---|
| OPADRY II series AMB | 9 |
| Water* | q.s. |

*Evaporates during processing.

§ As an alternative to sodium bisulfite, or in addition to sodium bisulfite, any one or more of butylated hydroxyanisole, butylated hydroxytoluene, ascorbic acid, citric acid, propyl gallate, ascorbyl palmitate, and alpha-tocopherol can be used.

Manufacturing Process:

1. Mix prasugrel hydrochloride, sodium bisulfite and fumed silica (first quantity), and sift through a #60 mesh, then sift the mixture again.

2. Geometrically blend the mixture of step 1 with other excipients, except fumed silica magnesium stearate.

3. Add fumed silica and magnesium stearate, and blend the mixture.

4. Compress the mixture into tablets.

5. Coat the tablets of step 4 with Opadry AMB dispersion in water.

6. Package the tablets of step 5 in a closed HDPE bottle containing a Sorb-It packet.

As an alternative to sodium bisulfite, or in addition to sodium bisulfite, any one or more of butylated hydroxyanisole, butylated hydroxytoluene, ascorbic acid, citric acid, propyl gallate, ascorbyl palmitate, and alpha-tocopherol can be used.

### Example 13

### Prasugrel Tablets

| Ingredient | mg/Tablet |
|---|---|
| Prasugrel hydrochloride | 65.88 |
| PEG 400 | 65.88 |
| Fujicalin ™§ | 98.82 |
| Croscarmellose sodium | 12 |
| HPMC 3 cps | 15 |
| Fumed silica | 6 |
| Magnesium stearate | 3 |

Coating

| | |
|---|---|
| OPADRY AMB | 9 |
| Water* | q.s. |

*Evaporates during processing.

§ Dibasic calcium phosphate anhydrous (spherically granulated), sold by Fuji Chemical Industry Co., Ltd., Japan. Alternatives for this ingredient include vegetable oil and glycerin.

Manufacturing Process:

1. Dissolve prasugrel hydrochloride in PEG 400 and mix with Fujicalin in a planetary mixer for 15 minutes.

2. Blend the croscarmellose sodium, HPMC 3 cps, fumed silica, and magnesium stearate with step 1.

3. Compress the mixture of step 2 into tablets.

4. Coat the tablets with an aqueous dispersion of Opadry AMB.

5. Package the tablets in a closed container with one or more of a molecular sieve, silica gel, and an oxygen absorber.

### Example 14

### Prasugrel Tablets

| Ingredient | mg/Tablet |
|---|---|
| Prasugrel hydrochloride | 65.88 |
| Lactose monohydrate | 131.76 |
| Lactose monohydrate | 64.71 |
| HPMC 3 cps | 10.5 |
| HPMC 3 cps | 4.5 |
| Stabilizing agent§ | 1.65 |
| Isopropyl alcohol* | q.s. |
| Croscarmellose sodium | 12 |
| Fumed silica (Aerosil R 972) | 6 |
| Magnesium stearate | 3 |

Coating

| | |
|---|---|
| OPADRY AMB | 9 |
| Water* | q.s. |

*Evaporates during processing.

§ One or more of butylated hydroxyanisole, butylated hydroxytoluene, ascorbic acid, citric acid, propyl gallate, ascorbyl palmitate, and alpha-tocopherol.

Manufacturing Process:

1. Blend prasugrel hydrochloride, lactose monohydrate (first quantity), HPMC 3 cps (first quantity) and croscarmellose sodium.

2. Compact the mixture using a roller compactor.

3. Mill compacted material through a 1.5 mm screen and pass through a #30 mesh sieve.

4. Granulate lactose monohydrate (second quantity), HPMC 3 cps (second quantity), and stabilizing agent with isopropyl alcohol in a rapid mixer granulator.

5. Dry the granules from step 4, mill through a 1.5 mm screen and pass through a #30 mesh sieve.

6. Mix compacted material of step 3 with granules of step 5 in a blender for 15 minutes, then blend with fumed silica and magnesium stearate.

7. Compress the blend of step 6 into tablets.

8. Coat the tablets with an aqueous dispersion of Opadry AMB.

1. A pharmaceutical formulation comprising prasugrel, or a pharmaceutically acceptable salt thereof, and at least one stabilizing agent.

2. The pharmaceutical formulation of claim 1, wherein a stabilizing agent comprises at least one of an antioxidant and a buffer.

3. The pharmaceutical formulation of claim 1, wherein a stabilizing agent comprises at least one of sodium metabisulfite, sodium bisulfate, sodium benzoate, and sodium sulfite.

4. The pharmaceutical formulation of claim 1, wherein a stabilizing agent comprises a citrate or phosphate buffer.

5. The pharmaceutical formulation of claim 1, in the form of a solid, liquid, semisolid, or paste.

6. The pharmaceutical formulation of claim 1, in the form of a solid tablet, capsule, tablet-in-tablet, mini-tablet, pellets, granules, or pellets compressed into a tablet, pellets filled into a capsule, granules compressed into a tablet, or granules filled into a capsule.

7. The pharmaceutical formulation of claim 6, in the form of a solid coated with at least one layer comprising a polymer.

8. The pharmaceutical formulation of claim 6, having a moisture content less than about 5 percent by weight.

9. The pharmaceutical formulation of claim 6, placed inside a package together with at least one of a desiccant and an oxygen scavenger.

10. The pharmaceutical formulation of claim 1, in the form of a liquid solution, viscous liquid, dispersion, suspension, or emulsion.

11. The pharmaceutical formulation of claim 1, in the form of a semi-solid ointment, paste, gel, or cream.

12. A pharmaceutical tablet comprising a salt of prasugrel and a stabilizing agent.

13. The pharmaceutical tablet of claim 12, wherein a salt of prasugrel comprises prasugrel hydrochloride.

14. The pharmaceutical tablet of claim 12, wherein a stabilizing agent comprises at least one of an antioxidant and a buffer.

15. The pharmaceutical tablet of claim 12, wherein a stabilizing agent comprises at least one of sodium metabisulfite, sodium bisulfate, sodium benzoate, and sodium sulfite.

16. The pharmaceutical tablet of claim 12, wherein a stabilizing agent comprises a citrate or phosphate buffer.

17. The pharmaceutical tablet of claim 12, having a moisture content less than about 5 percent by weight.

18. The pharmaceutical tablet of claim 12, placed inside a package together with at least one of a desiccant and an oxygen scavenger.

19. A pharmaceutical tablet comprising a salt of prasugrel and sodium bisulfite.

20. The pharmaceutical tablet of claim 19, wherein a salt of prasugrel comprises prasugrel hydrochloride.
